(19) United States
(12) Patent Application Publication
(10) Pub. No.: US 2016/0174910 A1
(43) Pub. Date: Jun. 23, 2016

(54) BIOLOGICAL EXERCISE INFORMATION DISPLAY PROCESSING DEVICE AND BIOLOGICAL EXERCISE INFORMATION PROCESSING SYSTEM

(71) Applicant: SEIKO EPSON CORPORATION, Tokyo (JP)

(72) Inventors: Hidetada KATO, Shiojiri (JP); Norimichi ITO, Azumino (JP); Kazuhiro SHIHO, Shiojiri (JP); Ayumu NAKAMURA, Matsumoto (JP)

(21) Appl. No.: 15/045,398

(22) Filed: Feb. 17, 2016

Related U.S. Application Data
(63) Continuation of application No. 13/240,008, filed on Sep. 22, 2011.

(30) Foreign Application Priority Data
Sep. 30, 2010 (JP) 2010-220810

(51) Int. Cl. A61B 5/00; G06F 3/01; A61B 5/11

(52) U.S. Cl. CPC A61B 5/744; A61B 5/1118; A61B 5/7475; A61B 5/4866; G06F 3/015

(57) ABSTRACT

A biological exercise information display processing device includes a biological information obtaining part, an exercise information obtaining part, a diet information obtaining part, a display character obtaining part, a display part, and a control part. The biological information obtaining part obtains biological information regarding a test subject. The exercise information obtaining part obtains exercise information regarding the test subject. The diet information obtaining part obtains diet information for the test subject. The display character obtaining part obtains a character representing the test subject. The display part displays the character. The control part determines a point based on the exercise information and the diet information, and when a given number of points has been accumulated, performs an exchange process to exchange the points for an item of the character.

### CROSS-REFERENCE TO RELATED APPLICATIONS

This application is a continuation application of U.S. patent application Ser. No. 13/240,008, filed on Sep. 22, 2011. This application claims priority to Japanese Patent Application No. 2010-220810 filed on Sep. 30, 2010. The entire disclosures of U.S. patent application Ser. No. 13/240,008 and Japanese Patent Application No. 2010-220810 are expressly incorporated by reference herein.

### BACKGROUND

1. Technological Field

The present invention relates to a biological exercise information display processing device and a biological exercise information processing system.

2. Background Technology

There have been disclosed biological exercise information display processing devices which display biological information, exercise information, or statuses that can be presumed therefrom with regards to the user or another person, in a manner that is readily understandable for non-expert members of the public; and which make it possible to create a variety of applications for purposes of health management or communication (e.g., see Patent Citation 1).

Japanese Patent Application Publication No. 2009-39157 (Patent Citation 1) is an example of the related art.

### SUMMARY

### Problems to be Solved by the Invention

However, with regards to guidance and support services intended to improve lifestyle so as to remediate/prevent metabolic syndrome or to maintain/improve health, an ordinary display method does not involve a character display that is linked to the status of progress of measures toward improvement to lifestyle, and therefore the status of progress cannot be grasped. Moreover, the characters are not set up by the user, and are therefore often lacking in a sense of reality or a sense of emotional attachment. As a result, there is a risk that the user will not be urged to maintain or increase their motivation or change their behavior, which are necessary in terms of improving lifestyle.

### Means Used to Solve the Above-Mentioned Problems

The invention has been devised in order to solve at least some of the above-mentioned problems, and can be realized according to embodiments or application examples described below.

According to one aspect of the invention, a biological exercise information display processing device comprises a biological information obtaining part, an exercise information obtaining part, a diet information obtaining part, a display character obtaining part, a display part, and a control part. The biological information obtaining part obtains biological information regarding a test subject. The exercise information obtaining part obtains exercise information regarding the test subject. The diet information obtaining part obtains diet information for the test subject. The display character obtaining part obtains a character representing the test subject. The display part displays the character. The control part determines a point based on the exercise information and the diet information, and when a given number of points has been accumulated, performs an exchange process to exchange the points for an item of the character.

According to the aspect of the invention, the biological exercise information display processing device further comprises a storage part that stores the biological information, the exercise information, the diet information, the point, and the item.

According to the aspect of the invention, the control part retrieves the biological information, the exercise information, the diet information, the point, and the item, and displays the character with the item on the display part.

According to the aspect of the invention, the biological information is information regarding at least one of a pulse rate, heart rate, height, weight, BMI, abdominal girth, body fat percentage, blood pressure, glycemia, cholesterol, neutral fat, GOT, GPT, and γ-GTP.

According to the aspect of the invention, the exercise information is information regarding at least one of an exercise time, number of steps taken, calorie expenditure, and exercise type.

According to the aspect of the invention, the diet information is at least one of calorie intake, diet content, meal times, diet volume, nutritional balance, and dietary routines.

According to the aspect of the invention, the item includes clothes for the character.

According to another aspect of the invention, a biological exercise information processing system comprises an information transmitting device and an information processing device. The information transmitting device includes a biological information detection part that detects biological information regarding a test subject, an exercise information detection part that detects exercise information regarding the test subject, and a transmitting part that transmits the biological information and the exercise information. The information processing device includes a communication part that obtains the biological information and the exercise information, a diet information obtaining part that obtains diet information for the test subject, a display character obtaining part that obtains a character representing the test subject, a display part that displays the character, and a control part that determines a point based on the exercise information and the diet information, and when a given number of points has been accumulated, performs an exchange process to exchange the points for an item of the character.

According to the aspect of the invention, the display part displays the character with the item.

According to the aspect of the invention, the biological information is information regarding at least one of a pulse rate, heart rate, height, weight, BMI, abdominal girth, body fat percentage, blood pressure, glycemia, cholesterol, neutral fat, GOT, GPT, and γ-GTP.

According to the aspect of the invention, the exercise information is information regarding at least one of an exercise time, number of steps taken, calorie expenditure, and exercise type.

According to the aspect of the invention, the diet information is at least one of calorie intake, diet content, meal times, diet volume, nutritional balance, and dietary routines.

According to the aspect of the invention, the item includes clothes for the character.

### BRIEF DESCRIPTION OF THE DRAWINGS

Referring now to the attached drawings which form a part of this original disclosure:

FIG. 1 is a block diagram of a biological exercise information detection display device according to the present embodiment;

FIG. 2 is a table showing conditions for determining the specifics of the expression/action of the character according to the present embodiment;

FIG. 3 is a flow chart showing the flow before the start of a lifestyle improvement program according to the present embodiment;

FIGS. 4A and 4B are drawings showing an illustration of a website screen according to the present embodiment;

FIG. 5 is a drawing showing the “street” screen for the character according to the present embodiment;

FIG. 6 is a block diagram of another example of the biological exercise information detection display device according to the present embodiment;

FIG. 7 is a flow chart showing the character image display process according to the present embodiment;

FIG. 8 is a drawing illustrating a biological exercise information processing system according to the present embodiment;

FIG. 9 is a block diagram of the biological exercise information transmitting device according to the present embodiment;

FIG. 10 is a block diagram of a server device according to the present embodiment;

FIG. 11 is a block diagram of the biological exercise information display device according to the present embodiment;

FIG. 12 is a block diagram of another example of the biological exercise information display device according to the present embodiment;

FIG. 13 is a flow chart showing the process of data transmission to the server device according to the present embodiment; and

FIG. 14 is a flow chart showing the process of transmitting data to the biological exercise information display device and performing a display according to the present embodiment.

### DETAILED DESCRIPTION OF EXEMPLARY EMBODIMENTS

A description will now be given for the present embodiment. The description will be given in the following sequence.

(Configuration of the biological exercise information detection display device)

(Character display process)

(System configuration)

(Configuration of biological exercise information transmitting device)

(Configuration of server device)

(Configuration of biological exercise information display device)

(System operation for character display)

(Effect and modification examples)

### (Configuration of Biological Exercise Information Detection Display Device)

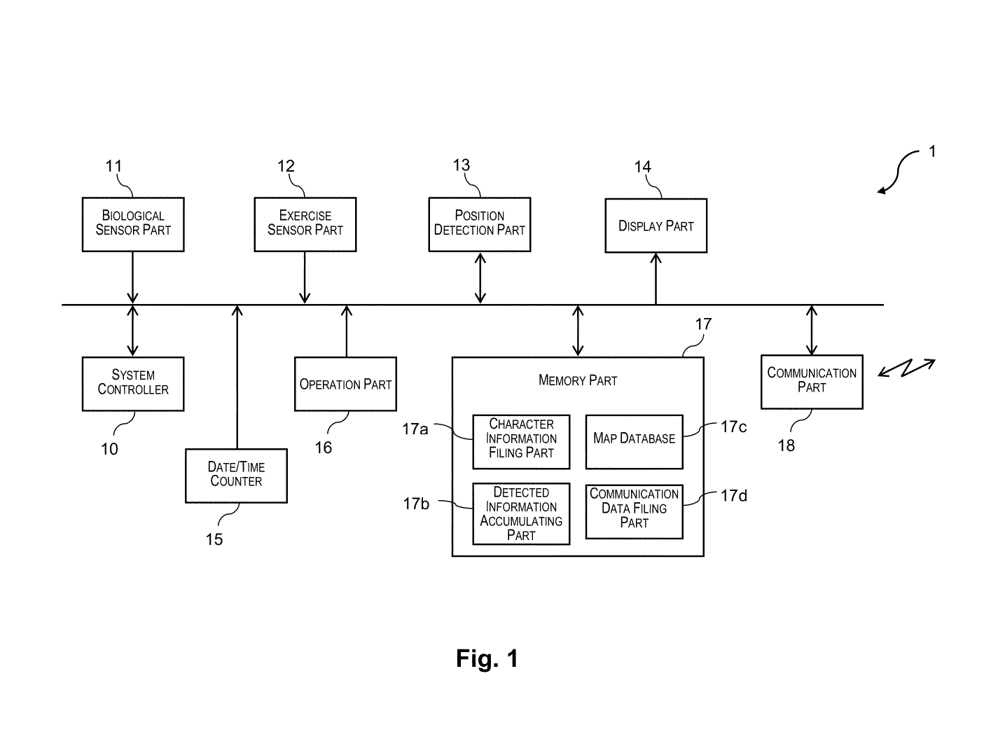

FIG. 1 is a block diagram showing a biological exercise information detection display device according to the present embodiment.

First, a configuration of a biological exercise information detection display device 1 will be described. The biological exercise information detection display device (biological exercise information display processing device) 1 of the present embodiment is a device carried by a user (test subject), and is an instrument for displaying, on its display screen, a character (i.e., a display character) based on biological information, exercise information, and diet information regarding the user who carries the device.

The biological exercise information detection display device 1 may be, e.g., an instrument that has a display worn by the user on the head like goggles, or one that is worn by the user on the arm as a wristwatch-type instrument. The biological exercise information detection display device 1 may also be an instrument having a shape and size suitable for portable use, similar to a mobile telephone handset or a personal digital assistant (PDA), carried around by the user. A portion having the function of the biological exercise information detection display device 1 may also be provided as a function installed on a mobile telephone or a PDA. The biological exercise information detection display device 1 may be employed by the user to measure biological information and exercise information during exercise or when the user is active within normal life. As for the information to be measured, the date and time of measurement, pulse rate, total exercise time, time over which the user is within the target pulse rate zone, number of steps taken, calorie expenditure, or other variables may be recorded.

The biological exercise information detection display device 1 has a system controller (control part) 10, a biological sensor part 11, an exercise sensor part 12, a position detection part 13, a display part 14, a date/time counter 15, an operation part 16, a memory part (storage part) 17, and a communication part 18.

The system controller 10 is configured from, e.g., a microcomputer including a central processing unit (CPU), a read-only memory (ROM), a random access memory (RAM), a non-volatile memory part, and an interface part; and is a control part for controlling the entire biological exercise information detection display device 1.

The system controller 10 controls various parts within the biological exercise information detection display device 1 based on an internal operation program, and executes a character display operation and other operations based on the biological information, the exercise information, and the diet information.

The system controller 10 includes a biological information obtaining part, an exercise information obtaining part, a diet information obtaining part, and a display character obtaining part. The biological information obtaining part obtains the biological information regarding the test subject. The exercise information obtaining part obtains the exercise information of the test subject. The diet information obtaining part obtains the diet information of the test subject. The display character obtaining part obtains the display character of the test subject. The system controller 10 determines the expression or the action of the display character based on the biological information, the exercise information, and the diet information; and performs a control so that an image display is performed on the display part 14 according to the expression or the action of the display character that has been determined.

The biological sensor part 11 detects the biological information regarding the user. Biological information refers to the pulse rate, heart rate, height, weight, BMI, abdominal girth, body fat percentage, blood pressure, glycemia, cholesterol, neutral fat, GOT, GPT, γ-GTP, and similar information regarding the user. The biological information regarding the test subject can thereby be readily obtained.

For example, a sensor that comes into contact with the skin of the test subject can be used to detect the pulse rate, blood pressure, or a similar variable. Biological information (BMI) is reflected in the character display or “body shape change.”

The biological sensor part 11 outputs the required information regarding the results of detection by the sensor to the system controller 10.

The exercise sensor part 12 detects the exercise information regarding the user. Exercise information refers to the exercise time, number of steps taken, calorie expenditure, exercise type, and similar information regarding the user. The exercise information regarding the test subject can thereby be readily obtained.

The exercise information can be detected using an acceleration sensor, a gyroscope (angular velocity sensor), a vibration sensor, or another sensor. Specifically, providing an acceleration sensor or a gyroscope makes it possible to detect, e.g., the movement of the whole body, head movement, neck movement, arm movement, leg movement, or another movement of the body as a signal corresponding to the movement of the user. It shall be apparent that, in an instance in which the movement of an arm or leg or some other event is to be detected, the acceleration sensor or the gyroscope is worn on the arm or the leg of the user.

The exercise sensor part 12 outputs information regarding results of detection by the acceleration sensor or another sensor to the system controller 10.

The position detection part 13 is, e.g., a GPS receiving part. The GPS receiving part receives radio waves from a Global Positioning System (GPS) satellite, and outputs information regarding latitude and longitude to the system controller 10 as the current position.

The position detection part 13 may also use Wireless Fidelity (WiFi) or a position information service provided by a mobile phone provider.

The date/time counter 15 performs a constant date/time-counting operation, and counts the year, month, day, hour, minute, and second.

The operation part 16 is provided as a key, a dial, or another operating element for the user of the biological exercise information detection display device 1 to perform a variety of operations. Alternatively, a touch panel may be provided to the display part 14, wherein the touch panel is capable of being operated by touch as the operation part 16.

For example, a power on/off operation, display-related operations (e.g., a display mode selection operation or a display adjustment operation), a variety of setting operations, a past status history display operation (described further below), operation, and similar operations are made possible.

The diet information regarding the user is inputted from the operation part 16. Diet information refers to the calorie intake, diet content, meal times, diet volume, nutritional balance, dietary routines, or similar information regarding the user. The diet information for the test subject can thereby be readily obtained. It shall be apparent that the biological information and the exercise information described above can also be inputted from the operation part 16.

The operation part 16 outputs, to the system controller 10, information regarding the results obtained through inputting as described above. The exercise information and the diet information are reflected in the character displayed on the “street” screen as the latest data. The character thereby performs an action according to the display and implementation status.

The system controller 10 performs the required control process based on operation information from the operation part 16.

Since the biological exercise information detection display device 1 according to the present embodiment includes the biological sensor part 11 and the exercise sensor part 12, conscious behavior on the part of the user may be ascertained from the biological information and the exercise information detected by the sensor parts, and judged by the system controller 10 as operation input information.

For example, a light tapping action applied by the user to the biological exercise information detection display device 1 can be detected by the acceleration sensor, the vibration sensor, or another sensor in the exercise sensor part 12, and recognized by the system controller 10 as an operation performed by the user.

Alternatively, a movement such as head-turning movement or a head-shaking movement performed by the user may be detected using the acceleration sensor or the angular velocity sensor and recognized by the system controller 10 as an operation performed by the user.

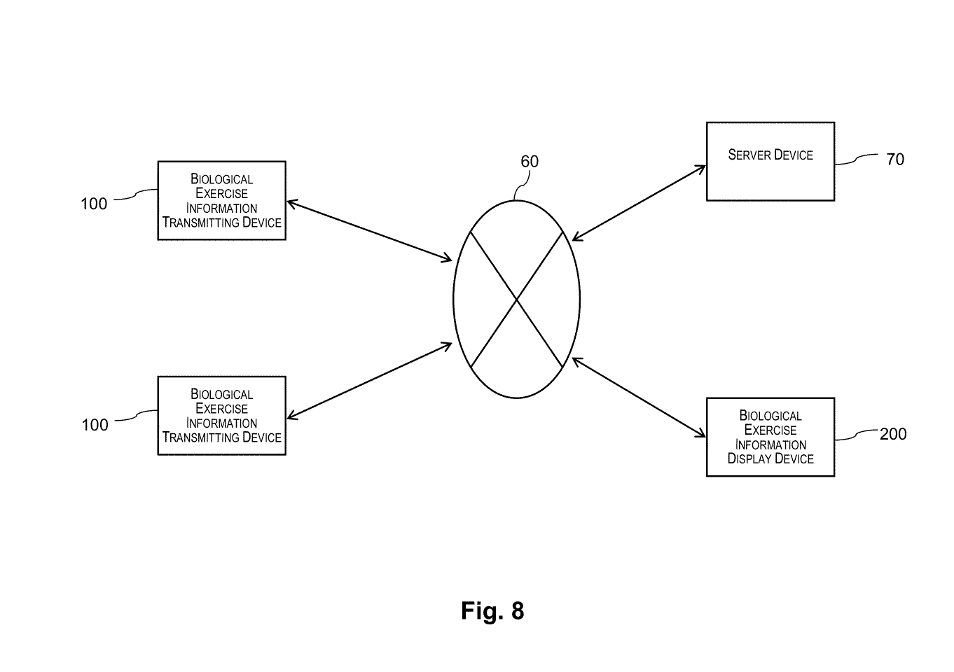

The communication part 18 performs transmission/reception of data with respect to an external instrument. The communication part 18 may have a wired or wireless network connection for communication. For example, the communication part 18 is configured as a portion for performing network communication in a system such as the one shown in FIG. 8 described further below or for performing direct data communication with other instruments.

The memory part 17 records (stores) a variety of types of data, or reproduces (retrieves) recorded data, based on a control performed by the system controller 10. Upon obtaining the biological information, the exercise information, and the diet information based on a control performed by the system controller 10, the memory part 17 stores the obtained biological information, exercise information, and diet information. Alternatively, the memory part 17 stores character-related information representing expressions or actions of the character determined based on the obtained biological information, exercise information, and diet information. It is thereby possible to store the biological information, the exercise information, and the diet information for the test subject for a certain period of time.

The system controller 10 retrieves the biological information, the exercise information, and the diet information from the memory part 17 under predetermined conditions, and performs display control of the display character using the biological information, exercise information, and diet information that has been retrieved. Alternatively, the system controller 10 retrieves the character-related information, and performs display control of the display character using the character-related information that has been retrieved. Since the biological information, the exercise information, and the diet information for the test subject is thereby stored for a certain period of time, by specifying a past point in time within the range in which the information is stored and causing the corresponding biological information, exercise information, and diet information to be extracted, it is possible to display the past status of the test subject using a display character.

The memory part 17 may be configured using a RAM, a flash memory device, or another solid-state memory device, or using, e.g., a hard disk drive (HDD).

The memory part 17 may also be a record/playback drive corresponding to a portable recording medium, e.g., a memory card on which a solid-state memory device is installed, an optical disk, an magneto-optical disk, a hologram memory device, or a similar recording medium, rather than an installed recording medium.

It shall be apparent that both a solid-state memory device, HDD, or another installed-type memory device; and a record/playback drive corresponding to a portable recording medium, may be furnished. The memory part 17 may be a server device described further below.

In the present embodiment, there is prepared in the memory part 17 a memory region used for, in particular, a character information filing part 17a, a detected information accumulating part 17b, a map database 17c, and a communication data filing part 17d.

Image data representing the character to be displayed is filed in the character information filing part 17a. For example, a variety of image data representing the expression of the character, the action of the character, or an image showing the character design are stored.

The detected information accumulating part 17b stores the biological information detected by the biological sensor part 11, the exercise information detected by the exercise sensor part 12, and the position information detected by the position detection part 13. For example, the information described above is stored at given time intervals, alongside the date/time information counted by the date/time counter 15, based on a control performed by the system controller 10.

In other words, the detected information accumulating part 17b accumulates a history of the biological information, the exercise information, and the position information regarding the user.

The map database 17c stores a map image and other information necessary for map display.

The communication data filing part 17d is used for buffering or filing of data relating to transmission/reception by the communication part 18.

The display part 14 is provided with, e.g., a liquid crystal panel, an organic EL panel, or another display panel part; and a display driving part for driving the display panel part so that displaying is performed. The display driving part is configured using a pixel driving circuit for causing supplied image data to be displayed by the display panel part. The pixel driving circuit applies, at predetermined horizontal/vertical driving timings, a driving signal based on a visual image signal for each of the pixels arranged in a matrix pattern on the display panel part; and causes displaying to be executed.

The display part 14 causes the display panel part to execute a predetermined display based on a control performed by the system controller 10. In particular, in the present embodiment, the system controller 10 feeds character image data that is based on the biological information, the exercise information, and the diet information to the display part 14, and causes the display part 14 to execute character display. The system controller 10 determines, from the biological information, the exercise information, and the diet information, the character image to be displayed; and feeds the character image to the display part 14.

There are also instances in which the system controller 10 causes the display part 14 to execute map display using the map image data stored in the map database 17c.

The biological exercise information detection display device 1 of such description is preferably configured as a compact and light instrument so as to be capable of being worn by the user. Depending on the type of biological information to be detected, an appropriate format is, e.g., a wristwatch-type, goggle-type, headset-type, hat-type, helmet-type, or glove-type instrument; or an article of clothing in which the instrument has been installed; and in particular, one in which a part of the instrument (i.e., the biological sensor part 11) can be brought into contact with the skin, the head, or another appropriate part of the test subject's body in accordance with the specifics of the detection.

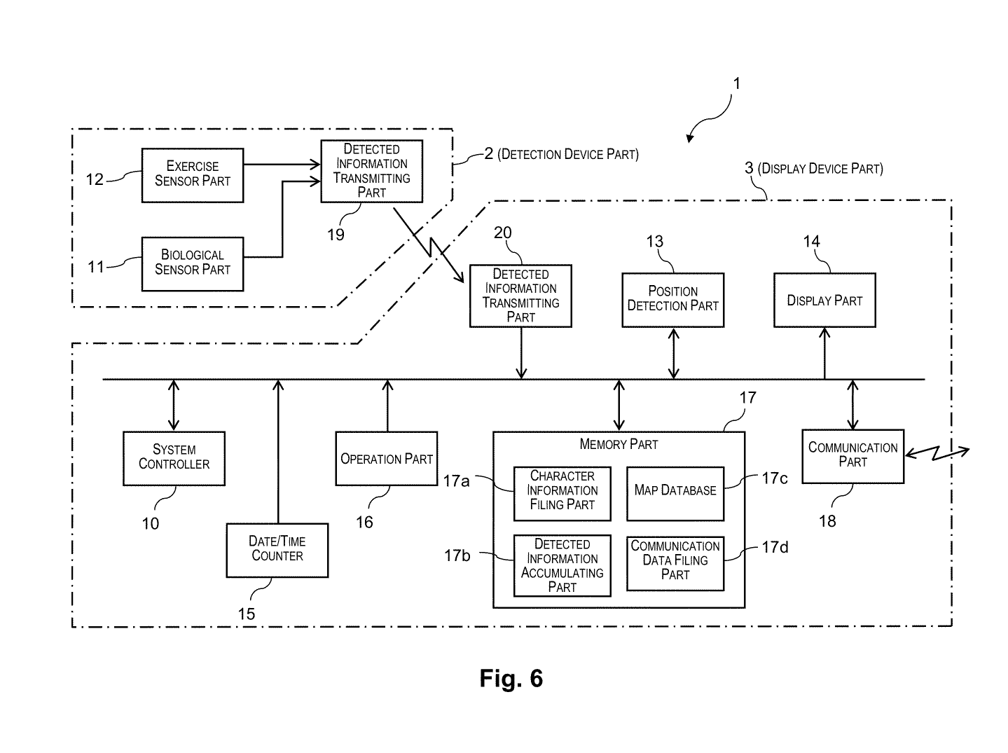

Since at least the biological sensor part 11 (or, the exercise sensor part 12 depending on the specifics of the exercise information to be detected) is required to come into intimate contact with the user's body, the biological exercise information detection display device 1 may be formed using two units as shown in FIG. 6 described further below.

A description will next be given for the character.

The biological exercise information detection display device 1 accumulates the biological information, the exercise information, and the diet information regarding the user, and displays a character that has been determined based on the above information. For example, the biological exercise information detection display device 1 displays, on the “street” screen, characters representing the user or another person.

The body shape of each of the characters changes according to the biological information (BMI) regarding the user. The action of each of the characters is determined by the exercise information and the diet information. For example, with regards to the exercise information, the character jumps in an incremental manner depending on the total daily exercise time within a predetermined target pulse rate zone. With regards to the diet information, the character is incrementally increased in illumination depending on whether the diet information has been inputted or whether the daily calorie intake target has been reached. The character goes into sleep mode if neither the exercise information nor the diet information is present. Also, for further clarity, the character performs a walking motion if, e.g., the exercise information regarding the user shows that the user has performed a walking exercise for a long period of time. The user thereby views their own and other people's implementation status, increasing the motivation to exercise further.

According to the present embodiment, the status of progress in improving lifestyle is obtained from the biological information, the exercise information, and the diet information for the test subject and displayed as a character set up by the test subject themselves so as to be readily personalizable and easy to understand. It is thereby possible for the test subject to review their own progress status in a realistic manner for encouragement or reflection, or to compare their own progress status with that of another person to boost competitiveness. The test subject is able to increase their motivation and change their behavior.

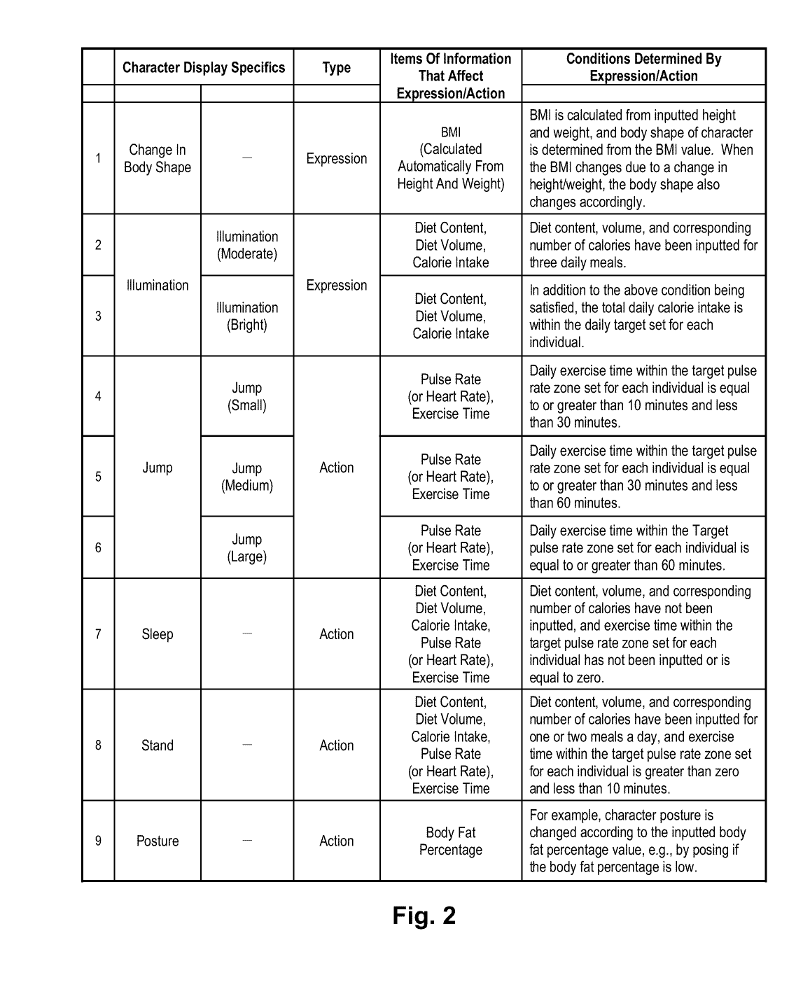

FIG. 2 is a table showing conditions for determining the specifics of the expression/action of the characters according to the present embodiment.

The specifics of character display include “body shape change” and “illumination” for expressions, and “jump”, “sleep”, and “stand” for actions. The conditions for determining the expression and action of the characters will now be described.

1. “Body shape change” is determined from the BMI calculated from the inputted height and weight. When the BMI changes due to a change in the height or weight, the body shape also changes accordingly.

2. “Illumination (moderate)” is determined in relation to the inputted diet content, diet volume, and calorie intake when the diet content, diet volume, and calorie intake in relation to three daily meals have been inputted.

3. “Illumination (bright)” is determined in relation to the inputted diet content, diet volume, and calorie intake when the diet content, diet volume, and calorie intake in relation to three daily meals have been inputted; and when the total daily calorie intake satisfies the daily calorie intake target set for each individual.

4. “Jump (small)” is determined when the inputted pulse rate (or heart rate) and exercise time show that the exercise time during which the user is within the target pulse rate zone set for each individual is 10 minutes or more and less than 30 minutes per day.

5. “Jump (medium)” is determined when the inputted pulse rate (or heart rate) and exercise time show that the exercise time over which the user is within the target pulse rate zone set for each individual is 30 minutes or more and less than 60 minutes per day.

6. “Jump (large)” is determined when the inputted pulse rate (or heart rate) and exercise time show that the exercise time over which the user is within the target pulse rate zone set for each individual is 60 minutes or more per day.

7. “Sleep” is determined in relation to the inputted diet content, diet volume, calorie intake, pulse rate (or heart rate), and exercise time when the diet content, diet volume, and calorie intake are not inputted, and the exercise time over which the user is within the target pulse rate zone set for each individual is either not inputted or is zero minutes.

8. “Stand” is determined in relation to the inputted diet content, diet volume, calorie intake, pulse rate (or heart rate), and exercise time when the diet content, diet volume, and calorie intake are inputted in relation to one or two meals a day, and the exercise time over which the user is within the target pulse rate zone set for each individual is greater than zero minutes and less than 10 minutes.

“Body shape change” may be determined so as to change according to the inputted abdominal girth.

“Posture” may be determined so as to change according to the inputted body fat percentage. For example, an arms-raised pose is adopted if the body fat percentage is low.

“Illumination (moderate)” may be determined in an instance in which, e.g., four or more of the inputted blood pressure, glycemia, cholesterol, neutral fat, GOT, GPT, and γ-GTP are within a reference value.

“Illumination (bright)” may be determined in an instance in which all of the inputted blood pressure, glycemia, cholesterol, neutral fat, GOT, GPT, and γ-GTP are within a reference value.

“Jump (small)” may be determined in an instance in which the inputted exercise time or the number of steps taken, or the calorie expenditure, shows that the number of steps taken per day is 5000 or more, or that the calorie expenditure is 100 kcal or more.

“Jump (medium)” may be determined in an instance in which the inputted exercise time or the number of steps taken, or the calorie expenditure, shows that the number of steps taken per day is 10,000 or more, or that the calorie expenditure is 300 kcal or more.

“Jump (large)” may be determined in an instance in which the inputted exercise time or the number of steps taken, or the calorie expenditure, shows that the number of steps taken per day is 15,000 or more, or that the calorie expenditure is 500 kcal or more.

“Posture” may be determined so that the character is displayed in a running posture if the inputted exercise type is “jogging,” and a walking posture if the inputted exercise type is “walking.”

“Illumination” may be determined so that a display will appear when the specifics of an inputted dietary routine (“no snacking” or the like) have been followed, and the fact that the routine has been implemented has been inputted (using a check box or another method).

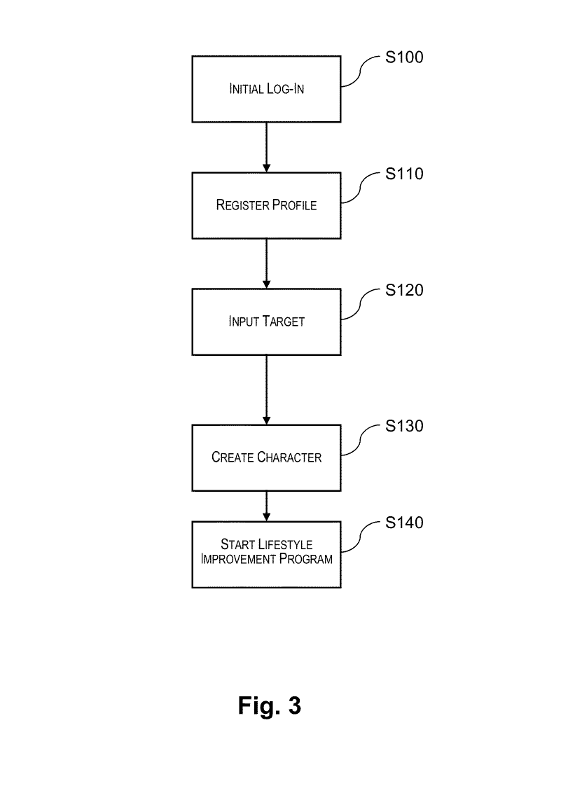

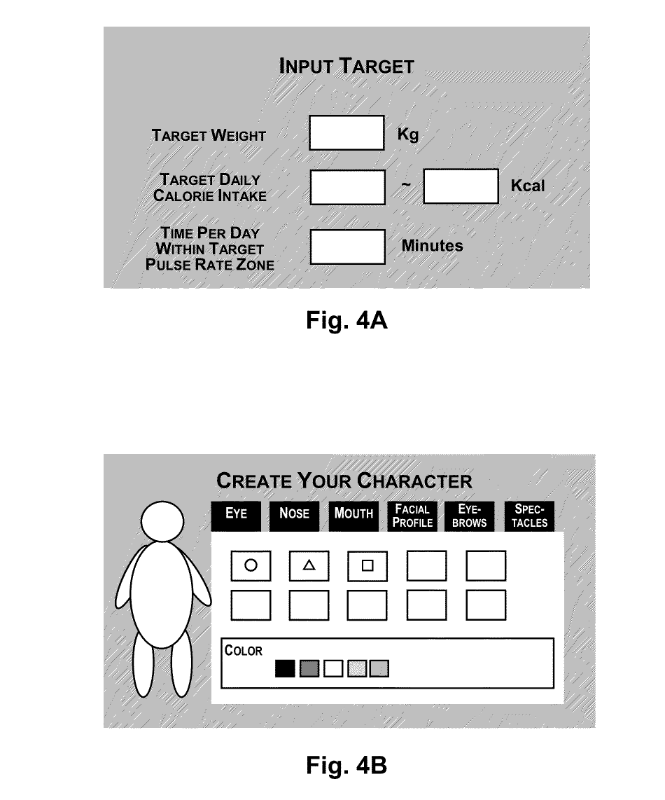

FIG. 3 is a flow chart showing the flow before the start of the lifestyle improvement program according to the present embodiment. FIGS. 4A and 4B are drawings showing an illustration of a website screen according to the present embodiment.

First, in step S100, the user logs in on the log-in screen using an ID and password.

Next, in step S110, the user inputs and registers personal information on the profile registration screen. Examples include name, nickname, gender, date of birth, height, weight, and life pattern.

Next, in step S120, the user inputs targets in the target input screen. For example, the user inputs a target weight as shown in FIG. 4A. A dietary target is also inputted.

A target value for the daily calorie intake is inputted. A value calculated using the following equation from the target weight and a coefficient related to life pattern is used as the target calorie intake value. An exercise target is inputted.

Target calorie intake(lower limit to upper limit)=target weight×coefficient related to life pattern

The coefficient related to life pattern is determined as follows. A value of 25 to 30 represents the user walking for about an hour for leisure or shopping, and otherwise remaining sedentary. A value of 31 to 35 represents the user walking, using public transport, dealing with customers, or performing housework for about two hours for commuting, for work, or for a similar purpose; and otherwise remaining sedentary. A value of 36 to 40 represents a user, whose coefficient related to life pattern would otherwise be 31 to 35, cycling or performing a similar exercise for about an hour a day. Standing activities, of which an hour involves heavy labor, represent the majority. A value of 41 to 45 represents strenuous training, carrying of wooden materials, farm work, or other strenuous activity performed for about an hour a day. A target value for the time over which the user is within the target pulse rate zone each day is also inputted.

Next, in step S130, the user creates their own character in the character creation screen. For the creation of the character, the body shape (BMI), the face (components selection), clothing (default), and other particulars are created. For example, the body shape is set according to the BMI. If the BMI is 30 or more, or 23 or less, the body shape does not change beyond a given extent. The face is created on the character setting screen by selecting the face shape, hairstyle, eyes, nose, mouth, eyebrows, ears, skin color, and other particulars. For example, as shown in FIG. 4B, in the creation of the face, the eyes, nose, mouth, facial profile, eyebrows, spectacles, and color are selected. The body shape of the character to be displayed is determined according to the BMI, calculated from the height and weight inputted in profile registration.

The characters are used to generate interest in the program among a poorly motivated demographic through fun and comparison with others, as with a game; and change behavior.

With the character “body shape change,” the body shape is changed according to the change in BMI since the start of the program. With regards to the character display, the character setting and the body shape change are displayed in order to generate, through fun, enthusiasm for participation in the program. With regards to the character display, the initial body shape is displayed as a silhouette so that it is easy to see the change in the body shape. For example, the initial body shape is visually expressed as being slightly thicker than reality.

Next, after the start of the lifestyle improvement program in step S140, the top page dedicated to the user is used to input data; view the character, various graphs, and rankings; or perform other operations.

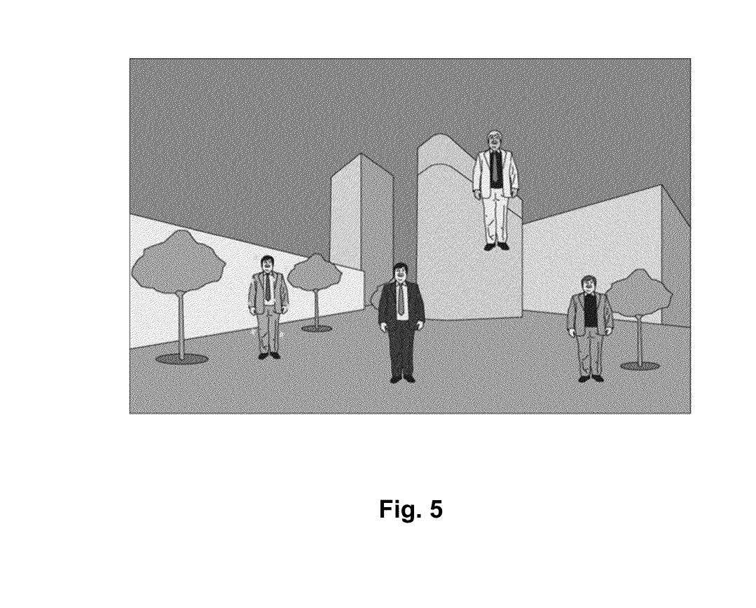

FIG. 5 is a drawing showing the “street” screen for the character according to the present embodiment.

The “street” screen displays characters representing the user and other people in order to generate changes in awareness and in behavior through comparison with others. It is possible to see who is making an effort, and who is not making an effort, with regards to exercise and diet. It is also possible to see other people's exercise implementation status. It is possible, on the “street” screen, to see who is making an effort.

The “street” screen shows avatars of the user and other people to poorly motivated people, who see their own avatar being inactive while other people are active, and generates motivation.

For the characters displayed on the “street” screen, the top ten performers in exercise/diet are selected and displayed each month from the ranking (wherein the best and poorest performers are displayed on the “street” screen). This is to prevent the displayed characters being solely poor performers, since the aim is to show those who are performing well. The names of the bottom ten performers who have not been exercising are selected and displayed on a monthly basis.

The number of avatars displayed in the “street” screen is preferably about ten to twenty including the user. This is because the screen will become difficult to see if a large number of avatars are shown. The user's own character is always displayed in the center.

For information regarding other people, when a character on the screen is clicked, the body shape change, target achievement rate, exercise implementation status, and other information regarding another person are displayed in another window. The user thereby sees exercise progress regarding other people for reference. For example, the information regarding other people may be the target daily exercise time (e.g., 30 minutes or more), or the exercise time over which the person is within the target pulse rate zone. The above only applies when the user sets the information to be available to others.

The user's own character may perform a jumping action depending on the time over which the user is within the target pulse rate zone according to the exercise information. For example, the time over which the user is within the target pulse rate zone according to the exercise information may be categorized into 10 minutes or above, 30 minutes or above, and 60 minutes or above, and the height of the jumping action varied between three levels.

The character may moderately increase in illumination when diet information in relation to three meals has been inputted. The character may, in addition, appear in bright illumination when the calorie intake is within the target calorie intake range.

“Illumination (moderate)” may be displayed when diet information in relation to three or more meals per day has been inputted, and “illumination (bright)” may be displayed around the user's own character when a person falls within the target calorie level.

Characters who have no record for either exercise information o diet information may adopt a lazy posture. In an instance in which neither the exercise information nor the diet information has been inputted, the character may adopt a lying posture.

The character may be displayed using, e.g., the status of improvement in blood components as biological information. Improvements within the body are thereby shown using character display even in an instance in which there has been no change in the body shape, helping to increase motivation.

The user's own character may reflect behaviors actually made by the person, and may make actions in the “street” screen for their own character. The action of the user's own character in the “street” screen may reflect data from three days beforehand.

In coordination with the ranking, a badge may also be displayed on a character in an instance in which the corresponding user is the poorest performer according to the ranking and given conditions are satisfied (e.g., weekly exercise time being under a certain number of minutes). Users whose character is wearing a badge are thereby identifiable to others as not exercising, and the users are therefore motivated so that the badge is no longer worn in the future.

Points may also be awarded according to exercise and diet status. The user can thereby understand their sustained progress from the number of points or items held by the character, where the items are purchased when a given number of points have been accumulated; and motivation for further effort can therefore be increased.

Points can be awarded according to the exercise information and diet information inputted, and exchanged for items for characters, in order to increase motivation by using incentives. The exercise information and the diet information can be reflected in the points awarded in the “street” screen as awardable-point data.

The character may also be downloaded as a screensaver. As a result, poorly motivated people who do not frequently log into the website are shown the screensaver and therefore reminded to improve their lifestyle even when they do not log in.

The screensaver is set so that those who do not log into the website are shown the state of the character and reminded to take measures regarding their exercise/diet.

The character is displayed on the screensaver. The body shape of the character changes according to the inputted data. There is a possibility that persons having poor motivation will not log into the website itself. Therefore, a screensaver on which the current state of the character is displayed in real time is created. The character is thereby displayed, even if the user is not logged into the website. Therefore, the user is allowed to check their own state in an objective manner, and thereby reminded of the program even when not logged into the website.

A check is made as to whether the environment allows the screensaver to be downloaded. The most up-to-date body shape is always displayed on the screensaver through the website and the server device. All participants are required to use the screensaver. The screensaver is automatically deleted when the program is discontinued.

FIG. 6 is a block diagram of another example of the biological exercise information detection display device according to the present embodiment. FIG. 6 shows an example in which the biological exercise information detection display device 1 is configured from separate units, which are a detection device part 2 and a display device part 3.

The detection device part 2 has the biological sensor part 11, the exercise sensor part 12, and a detected information transmitting part 19.

The display device part 3 includes a detected information receiving part 20 in addition to the system controller 10, the position detection part 13, the display part 14, the date/time counter 15, the operation part 16, the memory part 17, and the communication part 18.

The detected information receiving part 20 and the detected information transmitting part 19 communicate with each other by, e.g., wireless or wired communication.

In an instance in which wireless communication is used, Bluetooth or another short distance wireless communication scheme may be employed. Alternatively, an optical communication method, in which data communication is performed by optical pulse modulation using visible or invisible light, may be employed. It shall be apparent that network communication or a wireless communication scheme compatible with long distances may also be used.

In the instance shown in FIG. 6, biological information detected by the biological sensor part 11 and exercise information detected by the exercise sensor part 12 are transmitted from the detected information transmitting part 19 and received by the detected information receiving part 20. Diet information is inputted from the operation part 16.

The system controller 10 controls the character image display, based on the diet information inputted from the operation part 16 and the biological information and the exercise information received by the detected information receiving part 20.

According to the configuration shown in FIG. 6, only the detection device part 2 is required to come into contact with the user's skin, head, or another required portion. Therefore, a mode is possible in which the burden placed on the user in wearing the biological exercise information detection display device is reduced. In particular, the detection device part 2 has a simple configuration having the biological sensor part 11, the exercise sensor part 12, and the detected information transmitting part 19. Therefore, the size and weight can be readily reduced, and an instrument that can be worn on the required portion of the body is readily realized.

The display device part 3 may be a dedicated compact instrument carried by the user, or may also be realized as the function of the display device part 3 furnished on, e.g., a mobile telephone handset, a personal digital assistant (PDA), or another portable instrument. A usage configuration is possible in which the display device part 3 is not necessarily carried or worn by the user. In such an instance, the display device part 3 may be a relatively large device. The function of the display device part 3 may also be executed on a desktop-type or notebook-type PC or a similar device.

### (Character Display Process)

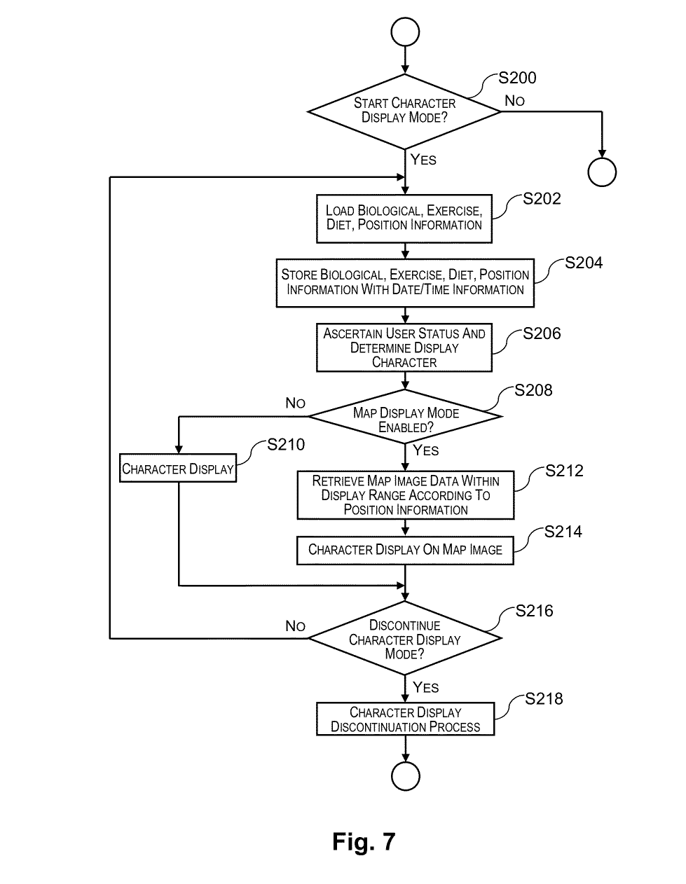

FIG. 7 is a flow chart showing the character image display process according to the present embodiment.

The character display process performed by the biological exercise information detection display device 1 will now be described with reference to FIG. 7. The display process described here is an operation in which the biological information, the exercise information, and the diet information are constantly detected; the character image is displayed on the display part 14 according to the detected information; and the status of the user is thereby expressed in real time.

For example, the biological exercise information detection display device 1 performs an operation for causing the display part 14 to display the status of the user when the character display mode has been enabled by an operation carried out by the user.

FIG. 7 shows a control process run by the system controller 10 to perform character image display.

When the character display mode is launched, the system controller 10 moves the process forward from step S200 to step S202, and launches the character image display process.

In step S202, the system controller 10 loads the biological information and the exercise information as information detected by the biological sensor part 11 and the exercise sensor part 12. The diet information inputted using the operation part 16 is also loaded. The position information detected by the position detection part 13 is also loaded.

Next, in step S204, the system controller 10 causes the detected information accumulating part 17b of the memory part 17 to store the loaded biological information, exercise information, diet information, and position information, together with the current date and time (in year, month, day, hour, minute, and second) counted by the date/time counter 15 at the corresponding point in time.

Next, in step S206, the system controller 10 ascertains the user status and determines the character, based on the loaded biological information, exercise information, and diet information.

The user status may be a status represented by the biological information, the exercise information, and the diet information themselves, or may be the user's psychological or emotional status inferred from the biological information, the exercise information, and the diet information.

In step S208, the process flow is separated according to whether the map display mode is currently enabled. The map display mode is a state in which the user has performed an operation so that map display is executed on the display part 14. For example, in an instance in which the user issues an instruction to enable the character display mode during map display or in an instance in which the user issues an instruction to display the map image while the process shown in FIG. 7 is being executed in character display mode, the map display mode is judged to be enabled in step S208.

If the map display mode is not currently enabled, the system controller 10 proceeds from step S208 to step S210 and performs character display control. Specifically, the system controller 10 feeds the character image data determined in step S206 to the display part 14 and causes the character image data to be displayed.

Meanwhile, if the map display mode is enabled, the system controller 10 proceeds from step S208 to step S212, and retrieves from the map database 17c, based on the current position information, the map image data relating to the range to be displayed. Then, in step S214, the system controller 10 generates the display image data from the map image data and the character image data; feeds the display image data to the display part 14; and executes display of the character on the map image. For example, the character image is displayed so as to express the user being present at a position that corresponds with the current position on the map image.

The system controller 10 returns to step S202 and repeats the same process until it is judged in step S216 that the character display mode has been discontinued.

In an instance in which, e.g., the user performs an operation to issue an instruction to discontinue the character display mode, the system controller 10 proceeds from step S216 to step S218, discontinues the character display on the display part 14, and ends the process shown in FIG. 7.

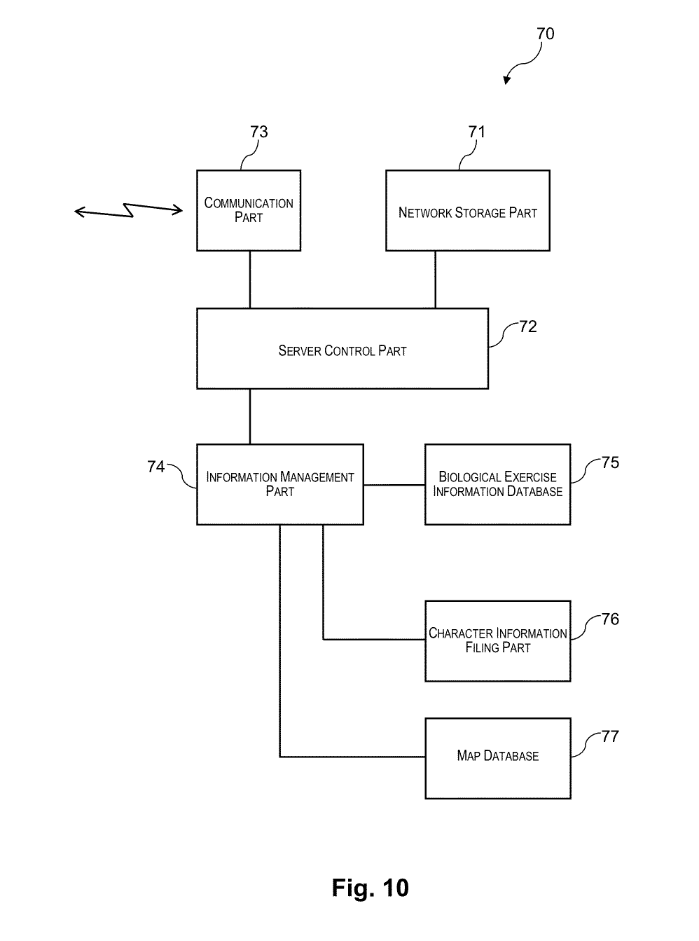

A specific character image may be prepared in advance and stored in the character information filing part 17a. However, e.g., communication may be performed with an external server device (e.g., a server device 70 described further below using FIG. 8, FIG. 10, and other drawings) using the communication part 18, so that the character image data can be downloaded.

It shall be apparent that a large number of core characters may be prepared in advance and stored so that the user can make a selection; alternatively, the user can create their own desired character.

### (System Configuration)

Operations for displaying a character image according to the current status of the user themselves or for reproducing and displaying a character image showing the past status of the user themselves using the biological exercise information detection display device 1 shown in FIG. 1 have been hitherto described. Next, a description will be given for a biological exercise information processing system using which the user can know the status of another person from a character image, and a device constituting such a system.

FIG. 8 is a drawing illustrating a biological exercise information processing system according to the present embodiment. FIG. 8 shows an example of a system configuration.

The biological exercise information processing system is an example of a system configured so that a biological exercise information transmitting device 100, the server device 70, and a biological exercise information display device 200 (biological exercise information display processing device) are capable of communicating with each other via a network 60.

The system is one in which the user of the biological exercise information display device 200 is able to know, according to a character image, the status of another person wearing the biological exercise information transmitting device 100.

A variety of types of network is possible for the network 60, such as the Internet, a mobile telephone communication network, a PHS communication network, an ad-hoc network, a LAN, or a similar network.

The biological exercise information transmitting device 100 is inputted with the diet information regarding the wearer; detects the biological information, the exercise information, the position information, and the date/time information; and transmits the information to the server device 70 using network communication. The transmission may, for example, be performed periodically.

The server device 70 accumulates the biological information, the exercise information, the diet information, the position information, and the date/time information, which has been transmitted, in an internal database. As described further below, identification information uniquely applied to the wearer of the biological exercise information transmitting device 100 (hereafter referred to as the “user ID”) is associated with the biological information and other information, and accumulates therewith.

The biological exercise information display device 200 is capable of using network communication to obtain the biological information, the exercise information, the diet information, the position information, and the date/time information regarding a specific user (another person) accumulated in the server device 70. The biological exercise information display device 200 determines the character image to be displayed based on the obtained biological information, exercise information, and diet information, or the user ID; and displays the character image. The position information and the date/time information may also be reflected when the display is presented.

Alternatively, the biological exercise information display device 200 may obtain, from the server device 70 and by network communication, the character image itself that is to be displayed; and display the character image.

The biological exercise information detection display device 1 having the configuration shown in FIG. 1 may function as the biological exercise information transmitting device 100 shown in FIG. 8, or may also function as the biological exercise information display device 200.

### (Configuration of Biological Exercise Information Transmitting Device)

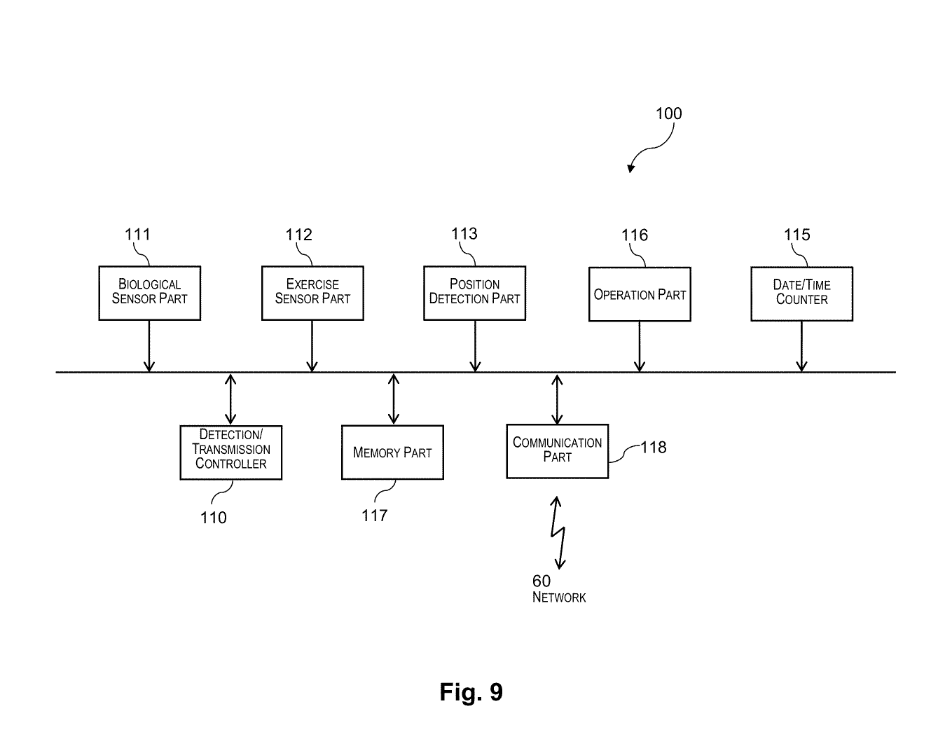

FIG. 9 is a block diagram of the biological exercise information transmitting device according to the present embodiment.

An example of the configuration of the biological exercise information transmitting device 100 shown in FIG. 8 above is shown in FIG. 9.

The biological exercise information transmitting device 100 has a detection/transmission controller 110, a biological sensor part (biological information detection part) 111, an exercise sensor part (exercise information detection part) 112, a position detection part 113, a date/time counter 115, an operation part 116, a memory part 117, and a communication part (transmitting part) 118.

The detection/transmission controller 110 is formed from, e.g., a CPU. The detection/transmission controller 110 performs a control process for the operation of detecting, or the operation of transmitting, biological information.

The memory part 117 has a ROM, a RAM, a non-volatile memory device, or another memory region; and is used as a region for filing process programs for the detection/transmission controller 110, as a work region, or for similar applications.

The memory part 117 may be configured as an internal memory for a microcomputer chip which functions as the detection/transmission controller 110.

Also, identification information uniquely associated with each individual biological exercise information transmitting device 100 (i.e., device ID) or identification information for the user of the biological exercise information transmitting device 100 (i.e., user ID) may be stored in, e.g., the non-volatile memory region of the memory part 117.

The communication part 118 transmits/receives data to/from an external communication instrument. Specifically, in the instance of the system configuration shown in FIG. 8, the communication part 118 performs data communication with the server device 70 in the form of communication via the network 60. In this instance, the communication part 118 may perform communication using a wireless or a wired connection to the network. An example is a configuration in which wireless communication is performed with a network access point.

The operation part 116 is provided for the user to perform the necessary operational input when the biological exercise information transmitting device 100 is to be used. For example, the operation part 116 may be configured so as to be capable of executing a power supply operation or a variety of setting operations.

The biological sensor part 111, the exercise sensor part 112, the position detection part 113, and the date/time counter 115 have the same function as the biological sensor part 11, the exercise sensor part 12, the position detection part 13, and the date/time counter 15 described for the biological exercise information detection display device 1 shown in FIG. 1; and detect the biological information, the exercise information, the position information, and the date/time information regarding the user. The diet information is inputted from the operation part 116.

In the biological exercise information transmitting device 100 of such description, the detection/transmission controller 110, e.g., periodically stores, in the memory part 117, the biological information, the exercise information, and the position information regarding the user detected by the biological sensor part 111, the exercise sensor part 112, the position detection part 113, and the date/time counter 115; the diet information inputted using the operation part 116; and the date/time information corresponding to the point in time of detection. Then, the biological information, the exercise information, the diet information, the position information, and the date/time information loaded into the memory part 117 is used to generate transmission data, which is transmitted from the communication part 118 to the server device 70 via the network 60. In this instance, the transmission data includes not only the biological information, the exercise information, the diet information, the position information, and the date/time information, but also the user ID, the device ID, and similar information.

The biological exercise information transmitting device 100 of such description is preferably configured as a compact and light instrument so as to be capable of being worn by the user in a convenient manner. Depending on the type of biological information, exercise information, and diet information to be detected, an appropriate configuration is, e.g., a wristwatch-type, goggles-type, headset-type, hat-type, helmet-type, or glove-type instrument; or an article of clothing installed with the instrument; and in particular, one in which a part of the instrument (i.e., the biological sensor part 111) can be brought into contact with the skin, the head, or another appropriate part of the test subject's body in accordance with the specifics of the detection.

Although not shown, the biological sensor part 111 and the exercise sensor part 112 may also be configured as separate units, as with the example shown in FIG. 6.

As can be understood by comparing the configuration shown in FIG. 9 and the configuration shown in FIGS. 1 and 6, the biological exercise information detection display device 1 described using FIGS. 1 and 6 may also perform the same action as the biological exercise information transmitting device 100 shown in FIG. 9 and function as the biological exercise information transmitting device 100 shown in FIG. 8. Specifically, e.g., the system controller 10 shown in FIG. 1 may generate, e.g., periodically, the transmission data using the biological information, the exercise information, the diet information, the position information, and the date/time information; and transmit the transmission data from the communication part 18 to the server device 70 via the network 60.

The biological exercise information transmitting device 100 may also be configured so that a target pulse rate zone (upper limit, lower limit), in which the exercise intensity is between 40% and 70% where fat is said to readily burn, can be set. The biological exercise information transmitting device 100 may use an LED, vibration, or an alarm on the instrument, or otherwise notify the user when the pulse rate enters the target pulse rate zone during exercise measurement.

### (Configuration of Server Device)

FIG. 10 is a block diagram of a server device according to the present embodiment.

Next, an example of the configuration of the server device 70 is shown in FIG. 10.

As described above, the server device 70 is a device that is capable of accumulating the biological information, the exercise information, the diet information, the position information, and the date/time information transmitted from the biological exercise information transmitting device 100 through communication via, e.g., the network 60; and of transmitting the accumulated biological information and other information to the biological exercise information display device 200.

The server device 70 includes a server control part 72, a network storage part (accumulating part) 71, a communication part 73, an information management part 74, and a biological exercise information database 75. The server device 70 may also include a character information filing part 76 and a map database 77.

The server control part 72 controls operations that are necessary for the server device 70.

Specifically, the server control part 72 performs control of the network communication operation; process control when the biological information, the exercise information, the diet information, the position information, and the date/time information have been transmitted from the biological exercise information transmitting device 100; and control relating to transmission of the biological information and other information to the biological exercise information display device 200.

The network storage part 71 is formed from, e.g., an HDD; and is used to, e.g., temporarily store transmission/reception data relating to communication with the biological exercise information transmitting device 100 or the biological exercise information display device 200 via the network 60, to store a variety of necessary data, or for similar uses.

The communication part 73 performs data communication via the network 60 with the biological exercise information transmitting device 100 or the biological exercise information display device 200.

The information management part 74 manages the biological information, the exercise information, the diet information, the position information, and the date/time information transmitted from the biological exercise information transmitting device 100.

The biological information, the exercise information, the diet information, the position information, and the date/time information transmitted from the biological exercise information transmitting device 100 is converted into a database format and filed in the biological exercise information database 75 in a state in which the information is associated with, e.g., a user ID or similar information.

The information management part 74 also performs data registration, searches, and similar processes with regard to the biological exercise information database 75.

The character information filing part 76 stores a variety of character images to be displayed on the biological exercise information display device 200. Specifically, the character image may be stored in a state in which it is associated with a user ID and managed.

Images and other necessary data for performing map display on the biological exercise information display device 200 are filed in the map database 77.

### (Configuration of the Biological Exercise Information Display Device)

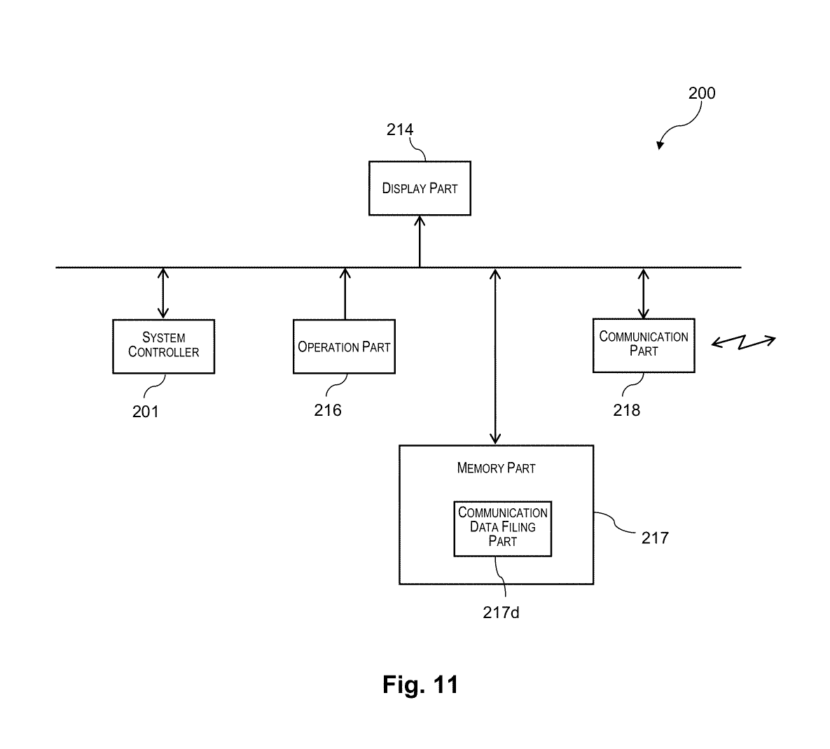

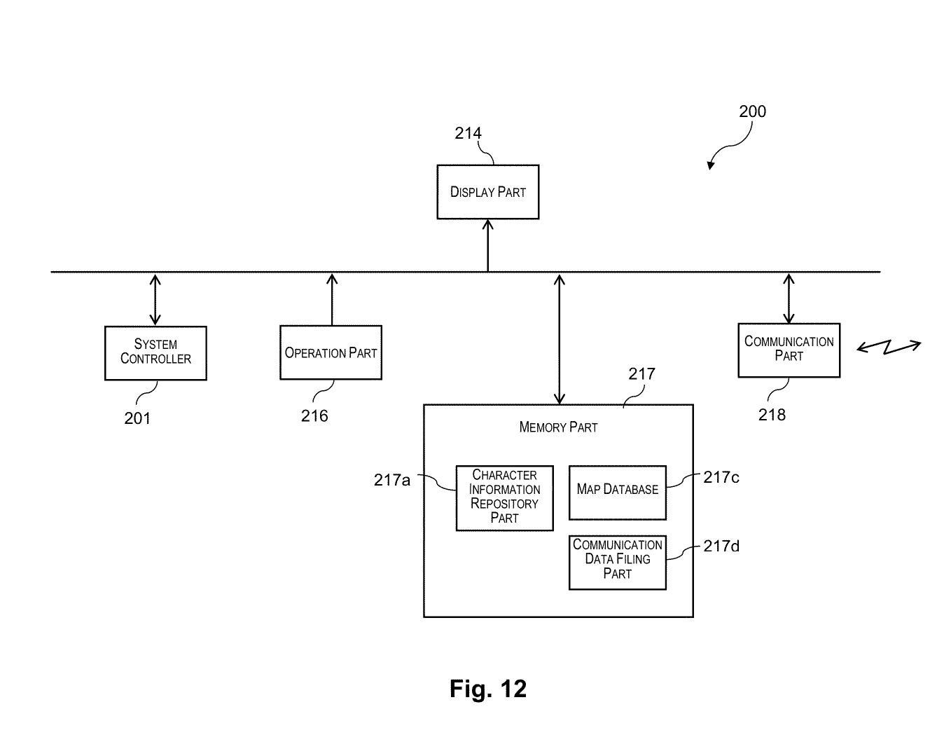

FIG. 11 is a block diagram of the biological exercise information display device according to the present embodiment. FIG. 12 is a block diagram of another example of the biological exercise information display device according to the present embodiment.

Next, examples of the configuration of the biological exercise information display device 200 will be described with reference to FIGS. 11 and 12.

The biological exercise information display device 200 shown in FIG. 11 is an example in which the biological exercise information display device 200 has a system controller (control part) 201, a communication part 218, a display part 214, an operation part 216, and a memory part 217. A communication data filing part 217d is present as a memory region in the memory part 217.

The example of the configuration of the biological exercise information display device 200 shown in FIG. 12 is an example in which, in addition to the configuration shown in FIG. 11, memory regions representing a character information filing part 217a and a map database 217c are formed in the memory part 217.

Each of the parts in the configurations shown in FIGS. 11 and 12 is the same as the system controller 10, the communication part 18, the display part 14, the operation part 16, and the memory part 17 shown in FIG. 1, and a duplicate description will not be provided.

Specifically, the communication part 218 performs data communication with the server device 70 in the form of communication via the network 60 shown in FIG. 8.

The system controller 201 also controls the character display operation and the process of communication with the server device 70.

The biological exercise information display device 200 mentioned with regards to the system configuration shown in FIG. 8 may be a device which displays, on the display part 214, a character image showing the status of a certain person (i.e., a user of the biological exercise information transmitting device 100, who is a different person from the user of the biological exercise information display device 200), based on the information received from the server device 70.

In order to achieve this, two types of configuration examples of the biological exercise information display device 200 can be envisaged, in consideration of the system operation.

As described in the present embodiment, a character image is an image for expressing the user status, judged based on the biological information, the exercise information, and the diet information.

Specifically, the character image to be displayed is determined based on the user status judged from the biological information, the exercise information, and the diet information. In an instance in which the process of determining the character image is considered a system operation, the process may be performed on the server device 70, or may be performed on the biological exercise information display device 200.

In an instance in which the character image to be displayed based on the user status judged from the biological information, the exercise information, and the diet information is determined on the server device 70, the server device 70 may transmit, to the biological exercise information display device 200, the data corresponding to the character image to be displayed. In such an instance, the biological exercise information display device 200 may stores the received character image data in the communication data filing part 217d, and then use this character image data to perform the display process; and can therefore be realized as the configuration shown in FIG. 11.

In an instance in which the character image to be displayed based on the user status judged from the biological information, the exercise information, and the diet information is determined on the server device 70, the server device 70 may transmit, to the biological exercise information display device 200, information that specifies the character image to be displayed, or information that specifies the expression or the action of the character to be displayed, instead of the data that corresponds to the character image to be displayed.

In such an instance, the biological exercise information display device 200 selects the character image and performs the display process based on the received information. Therefore, the configuration shown in FIG. 12, in which the biological exercise information display device 200 includes the character information filing part 217a, is appropriate.

In an instance in which a map image display is taken into account, if the map image data is to be downloaded from the server device 70 to the biological exercise information display device 200, the biological exercise information display device 200 may store the downloaded map data in the communication data filing part 217d, and then use this map data to perform the display process. Therefore, the biological exercise information display device 200 can be realized as a configuration in which the map database is not provided, as shown in FIG. 11.

In an instance of a system operation in which the process of determining the character image is performed on the biological exercise information display device 200, the configuration shown in FIG. 12 is appropriate.

In such an instance, the server device 70 transmits at least the biological information, the exercise information, and the diet information regarding a certain person (user ID) to the biological exercise information display device 200. The determining of the character image to be displayed based on the user status judged from the received biological information, exercise information, and diet information, and the performing of the display process, take place on the biological exercise information display device 200. Therefore, a configuration in which the character information filing part 217a is present is required.

As can be seen from the description hitherto given, the configuration shown in FIG. 11 is an example of a configuration that aims to simplify the biological exercise information display device 200, assuming a system operation in which the data representing the character image to be displayed is transmitted from the server device 70; and the configuration shown in FIG. 12 is an example of a configuration that can be employed irrespective of whether the process of determining the character is performed on the server device 70 or the biological exercise information display device 200.

Similarly, the biological exercise information detection display device 1 shown in FIG. 1 is an example of a configuration in which the biological exercise information detection display device 1 can function as the biological exercise information display device 200 irrespective of where the process of determining the character is performed.

### (System Operation for Character Display)

A description will now be given for an example of an operation of a biological exercise information processing system configured from the biological exercise information transmitting device 100, the server device 70, and the biological exercise information display device 200 described above.

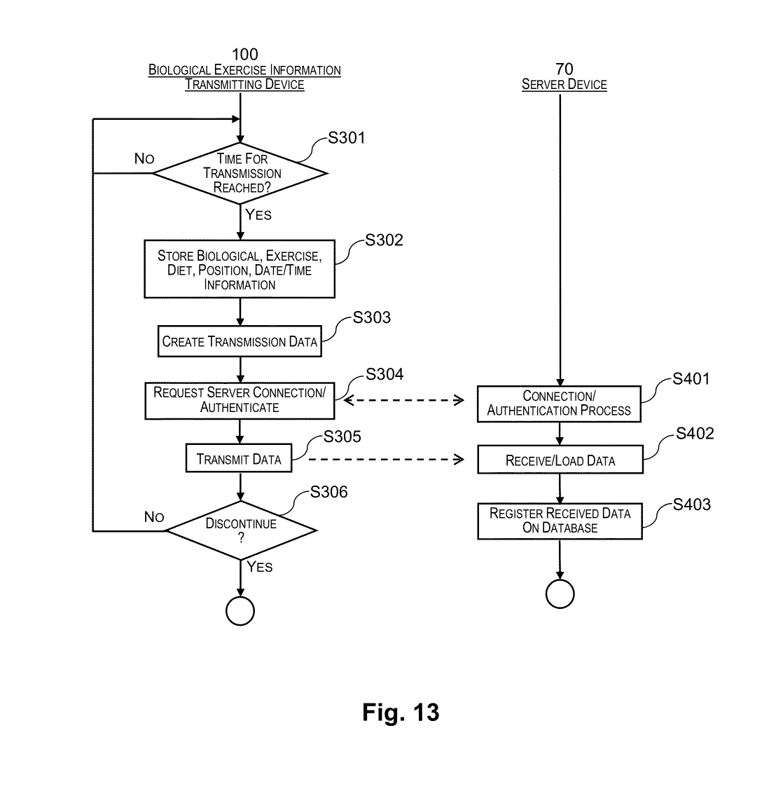

FIG. 13 is a flow chart showing the process of data transmission to the server device according to the present embodiment. FIG. 13 shows the operation when the biological information and other information is to be transmitted from the biological exercise information transmitting device 100 to the server device 70. In FIG. 13, the process performed by the biological exercise information transmitting device 100 is a process executed according to a control performed by the detection/transmission controller 110, and the process performed by the server device 70 is a process executed according to a control by the server control part 72.

In the biological exercise information transmitting device 100, the detection/transmission controller 110 stands by for the time to transmit in step S301. When the time for transmission is reached, the flow proceeds to step S302. The transmission may be performed at, e.g., a periodic timing, or a timing based on an operation performed by the user or another trigger.

In step S302, the detection/transmission controller 110 loads the biological information obtained by the biological sensor part 111; the exercise information obtained by the exercise sensor part 112; the biological information, the exercise information, and the diet information inputted at the operation part 116; the position information obtained by the position detection part 113; and the current date/time information obtained by the date/time counter 115; and stores the above information in the memory part 117.

Next, in step S303, the detection/transmission controller 110 generates the transmission data. Specifically, the detection/transmission controller 110 generates data for transmission that includes the user ID or the instrument ID in addition to the biological information, the exercise information, the diet information, the position information, and the date/time information loaded to the memory part 117.

In step S304, a communication connection is established with the server device 70. The detection/transmission controller 110 commences network communication from the communication part 118, and causes the server device 70 to execute a communication connection. At the same time, on the server device 70, the server control part 72 performs, as step S401, a process of establishing a communication connection at the communication part 73, and an authentication process. A variety of schemes can be envisaged for the authentication process. For example, a possible scheme is one in which the device ID is transmitted from the biological exercise information transmitting device 100, and the server device 70 judges whether the corresponding device ID is one that has been correctly registered.

When authentication is successful and a connection is established, the biological exercise information transmitting device 100 performs data transmission. Specifically, the detection/transmission controller 110 transmits, from the communication part 118, data including the biological information, the exercise information, the diet information, the position information, the date/time information, and the user ID (or instrument ID).

Meanwhile, the server control part 72 of the server device 70 loads data received by the communication part 73 into the network storage part 71 as step S402.

When loading of the received data is complete, the server control part 72 performs, in step S403, decoding processing and data extraction with regard to the received data that has been loaded, and performs a process in which the biological information, the exercise information, the diet information, the position information, the date/time information, and the user ID (or the instrument ID) included in the received data is transferred to the information management part 74 and registered in the biological exercise information database 75.

On the biological exercise information transmitting device 100, the flow returns from step S306 to step S301 and performs the processes of steps S302 to S305 each time, e.g., a periodic transmission timing is reached, until the operation is discontinued (e.g., the power is switched off).

The process described above is, e.g., performed in a successive manner, whereby, on the server device 70, information transmitted from each of the biological exercise information transmitting devices 100 is accumulated in the biological exercise information database 75.

Based on the information thus accumulated in the server device 70, the biological exercise information display device 200 is able to display to the user of the biological exercise information display device 200 the status of another user, using a character image.

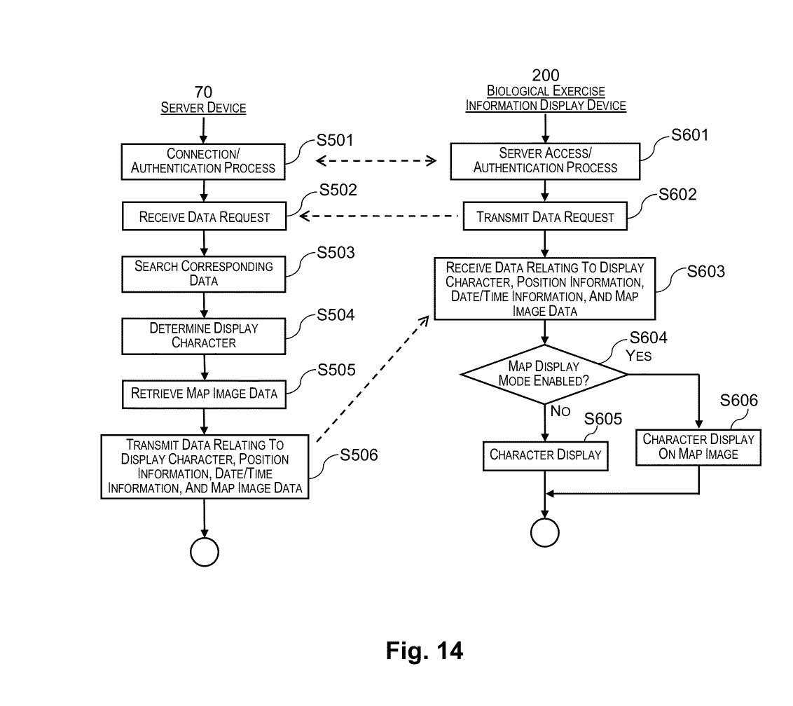

An example of a process for performing this display is shown in FIG. 14.

FIG. 14 is a flow chart showing the process of transmitting data to the biological exercise information display device and performing a display according to the present embodiment. FIG. 14 shows a process performed by the server device 70 and the biological exercise information display device 200. The process performed by the server device 70 is a process performed according to a control by the server control part 72, and the process performed by the biological exercise information display device 200 is a process performed according to a control by the system controller 201.

FIG. 14 is an example of the process in an instance in which the process of determining the character is performed in the server device 70.

First, the example of the process shown in FIG. 14 will be described.

In an instance in which the user of the biological exercise information display device 200 is aiming to know the current status of another person (e.g., an acquaintance), the user performs an operation for specifying the user ID (or the device ID) corresponding to the other person, and issues an instruction to access the server. For example, if the user ID of the other person is known, specifying this user ID makes it possible to perform an operation to request the status of the other person to be displayed.

In accordance with the user performing an operation of such description, the biological exercise information display device 200 performs the process shown in FIG. 14 in order to execute character display representing the status of the other person.

First, a communication connection is established with the server device 70 as step S601. The system controller 201 commences network communication from the communication part 218, and causes the server device 70 to execute a communication connection. At the same time, on the server device 70, the server control part 72 performs, as step S501, a process of establishing a communication connection at the communication part 73, and an authentication process. In this instance too, a variety of schemes can be envisaged for the authentication process. For example, a possible scheme is one in which the user ID of the user of the biological exercise information display device 200, or the device ID, is transmitted from the biological exercise information display device 200, and the user ID or the device ID is checked on the server device 70.

When communication with the server device 70 results in a successful authentication and communication is established, the system controller 201 of the biological exercise information display device 200 transmits to the server device 70, in step S602, a data request including the user ID (or the device ID) of the other person specified by the user of the biological exercise information display device 200.

When the server control part 72 detects, in step S502, that the data request has been received, the server control part 72 notifies the information management part 74 of the user ID (or the device ID) in step S503, and instructs the information management part 74 to search for the corresponding data. The information management part 74 extracts, from the biological exercise information database 75, data (i.e., the biological information, the exercise information, the diet information, the position information, and the date/time information) corresponding to the specified user ID (or device ID). When this happens, in an instance in which a plurality of registration data units have been accumulated (the biological information, the exercise information, the diet information, the position information, and the date/time information representing one registration data unit), the biological information, the exercise information, the diet information, the position information, and the date/time information representing the most up-to-date registration data unit may be retrieved according to the date/time information DATE.

Next, in step S504, the server control part 72 causes the information management part 74 to execute a process for determining the character.

The information management part 74 ascertains the user status using the biological information, the exercise information, and the diet information in particular, in relation to the biological information, the exercise information, the diet information, the position information, and the date/time information that has been retrieved. Then, the information management part 74 determines, from among the various character images stored in the character information filing part 76, the character image that represents the expression or the action corresponding with the ascertained user status.

At this time, if different character images have been prepared in advance for each of the user IDs, then the user ID is also used when determining the character image.

In step S505, the server control part 72 instructs the information management part 74 to retrieve the map image data. Based on the position information PL retrieved from the biological exercise information database 75, the information management part 74 retrieves, from the map database 77, the map image data within the necessary range including the position indicated by the image information PL.

In step S506, the server control part 72 receives, from the information management part 74, data relating to the determined character (e.g., the character image data itself), the position information PL and the map image data, and the date/time information DATE; and causes the received data to be transmitted from the communication part 73 to the biological exercise information display device 200.

In an instance in which the biological exercise information display device 200 has the configuration shown in FIG. 11, it is suitable for the data relating to the character to be the character image data. However, in an instance in which the biological exercise information display device 200 holds the character image as with the configurations shown in FIGS. 12 and 1, the data relating to the character may be information that specifies the character image or information regarding the ascertaining of the user status for specifying the character image.

On the biological exercise information display device 200, the system controller 201 performs, in step S603, the process of receiving the data relating to the character, the map image data, the position information PL, and the date/time information DATE. Specifically, the system controller 201 stores the above data received by the communication part 218 in the communication data filing part 217d of the memory part 217.

The system controller 201 then performs character image display based on the received/stored data.

In this instance, the system controller 201 checks, in step S604, whether the map display mode is currently enabled. This is the same as the process described for step S208 in FIG. 7.

If the map display mode is not enabled, the display part 214 is caused to execute character image display in step S605. Specifically, the received/stored character image data is fed to the display part 214, which is caused to execute character display.

If the map display mode is enabled, the display part 214 is caused to execute character image display on the map image in step S606. Specifically, the received/stored character image data and the map image data is fed to the display part 214, which is caused to execute character display.

In this instance, the user of the biological exercise information display device 200 is able to know, according to the character image, the current status (status based on the most up-to-date biological information, exercise information, and diet information accumulated in the server device 70) of their acquaintance.

Performing the above system operation allows the user of the biological exercise information display device 200 to know, from the character image display, the current status of another person who is wearing the biological exercise information transmitting device 100.

For example, the user of the biological exercise information display device 200 can recognize, from the character image, a variety of statuses regarding an acquaintance or another person, such as whether they are currently running or are feeling unwell. Also, it is possible for a mentor to analyze the data and guide each user in a manner that suits each individual. For example, the mentor refers to the data and encourages, praises, or otherwise guides the user.

According to the description above, the current status of another person is displayed using the character image. However, the past status of another person can also be displayed.

For example, if the biological information and other information regarding the user of the biological exercise information transmitting device 100 is stored for a certain period of time in the biological exercise information database 75 of the server device 70, by specifying a past point in time within the range in which the information is stored and causing the corresponding biological information and other information to be extracted, it is possible to display the past status of another person using a character image.

Also, in this example, the user of the biological exercise information display device 200 is able to see the status of another person whose user ID is known as for an acquaintance or due to a similar relationship. However, it is also possible to allow the status of a complete stranger, a celebrity, or a similar person to be seen using character display. For example, a configuration is possible in which a particular person consents to publicly disclose their own status, and registers on the system as such, whereby the user of the biological exercise information display device 200 is able to cause the biological exercise information display device 200 to display the status of the person who has consented to have their details disclosed, as desired.

The user may initially create their own character on the network. The character may be created by combining facial components (i.e., eyes, nose, mouth, facial profile, hairstyle, and similar components) in any manner. Since it is thereby possible to create a character according to individual choice, the user develops an emotional attachment to the character, and the user can gain enjoyment from the body shape change described below, or from a comparison between themselves and other people. This can inspire the user to persevere.

The body shape of the character may change according to the biological information (BMI value) inputted into the network by the user. The body shape of the character thereby improves due to a decrease in the BMI if lifestyle improvement is progressing well, making it possible for the user to gain a sense of achievement or encouragement upon viewing the improving body shape. Also, displaying characters representing other people generates enjoyment in viewing a variety of characters; and competitiveness arising from comparisons with other people, such as when the user is performing better than another person or is performing at a level that should be sustained regardless; making it possible to inspire the user to persevere. In Patent Citation 1, the height, weight, body fat percentage, and BMI are not items that are included as biological information.

The action/expression of the character may be displayed according to the biological information/exercise information instrumentally measured by the user and the diet information inputted into the network. Characters representing the user and other people can thereby be viewed.

The progress status of lifestyle improvement can be displayed using a character in a manner that is easy to understand so that anybody viewing the character can visually gain an understanding of the progress status. For example, with regards to the biological information/exercise information, the character can be made to jump in an incremental manner according to the total daily exercise time within a predetermined target pulse rate zone.

Also, with regards to diet information, the character may be displayed so as to incrementally increase in illumination according to whether the daily target calorie intake has been achieved.

The character may adopt a sleeping posture if neither the exercise information nor the diet information has been inputted. It is thereby possible for the user to view the action/expression of the character to understand the progress status regarding lifestyle improvement behavior (i.e., exercise or diet), allowing the user to self-review and reflect, such as when a user who did not achieve anything on a given day is inspired to perform in the future; when a user can see that although they did not exercise, they have performed well with regards to the diet; or when a user who sees their own character constantly sleeping is inspired to exercise.

The action/expression of the character and the body shape change may also be combined.

The user can thereby recognize the correlation between lifestyle improvement behavior (i.e., exercise and diet) and effect, appreciate the effectiveness of their actions, and gain encouragement and inspiration to continue to persevere. Also, the user can make a comparison with the action/expression of characters representing other people, and gain satisfaction from performing better than other people, or feel regret from not making sufficient effort or embarrassment from being seen to be doing nothing (i.e., the character is seen to be sleeping), encouraging the user to change behavior. In Patent Citation 1, a person's action status, health status, emotions, and similar statuses are displayed. However, the status of progress with regards to lifestyle improvement measures cannot be viewed, and a change in behavior does not ensue.

A method of comparing oneself with others to generate competitiveness which results in motivation is a ranking, where users are ranked using an indicator converted to numerical values. However, although this method motivates users who are ranked higher, users who are ranked lower are not motivated due to reasons such as an excessively wide gap between themselves and those in higher ranks or an aversion to being compared with others. Using characters for an indirect comparison or an expression that values individuality and that is easy to understand, rather than a direct and mechanical comparison using numerical values such as ranking, makes it possible to motivate particularly users who are poorly motivated, and cause them to change their behavior.

Points may also be awarded according to inputting of exercise information/diet information. Points may be exchanged for items for characters (such as clothes) when a given number of points has been accumulated, where the obtained items can be placed on the character as desired. The user can thereby view the appearance of characters representing other people, appreciate the efforts of other people who hold a large collection of items, gain interest in obtaining particular items, and be inspired to persevere. Also, an incentive is awarded for effort, resulting in enjoyment and encouragement. Patent Citation 1 does not describe incentives or changes to the appearance of characters.

Lifestyle-improving motivation can be inputted and displayed using the character. Other people's enthusiasm can thereby be viewed, which is associated with motivation for lifestyle improvement.

It is possible to communicate with other people through the characters. Information is thereby exchanged between those who are aiming to improve their lifestyle, and motivation is increased.

Position information obtained using GPS can be combined so that courses/locations of exercise performed by the user themselves and other people can be viewed together with the pulse rate data. Motivation for exercise is thereby increased, and a source of reference when exercise is performed is provided.

Points may also be awarded according to exercise and diet implementation status. The user can thereby understand sustained progress made by themselves or others from the number of points or items held by the character, where the items are purchased when a given number of points have been accumulated. Motivation for further effort can thereby be increased.

Exercise information regarding a character representing another person within a given period of time may be made viewable by clicking the character representing the other person.

It is thereby possible to see when and what time of day at which the other person performs exercise, and the amount of exercise performed by the other person, which the user can use as a reference in terms of their own exercise. It is possible to view information about people who have consented to having their information publically disclosed.

### Effect and Modification

According to the above embodiment, the user of the biological exercise information detection display device 1 (biological exercise information display device 200) can recognize their own status as well as that of other people through expressions and actions of characters. It is thereby possible to readily know their own status as well as that of other people in an enjoyable manner.

For example, a character display of a user's own status makes it possible to enjoy the display image.

Displaying a replayed character to display prior status makes it possible for the user to faithfully recall their own behavior or enjoy pleasant memories.

Also, by generating a character display of another person's status, the user can faithfully ascertain another person's status.

For example, the user can know the status or the current position of a friend who is running late for an appointment. Also, a parent can check the current position or the status of a child. Also, the status of a health-impaired person when they are not at home can be readily checked by a family member or another person. A variety of applications such as those described above can be imagined.

The above embodiments are not provided by way of limitation, and a variety of modifications can be envisaged as examples of device configurations or processes.

The above-described configuration of each of the biological exercise information detection display device 1, the biological exercise information transmitting device 100, the biological exercise information display device 200, and the server device 70 is merely given by way of example; it being apparent that a variety of constituent elements can be added or removed according to the actual example of operation or function to be performed.

The biological exercise information processing system shown in FIG. 8 is configured so that the biological exercise information display device 200 obtains, via the server device 70, the biological information and other information detected in the biological exercise information transmitting device 100. However, a system configuration in which the server device 70 is not required is also possible. Specifically, it is also possible to use a system in which the biological exercise information transmitting device 100 and the biological exercise information display device 200 directly communicate with each other, wherein the biological exercise information display device 200 receives the biological information, the exercise information, the diet information, the position information, and other information detected by the biological exercise information transmitting device 100, whereby character display based on the biological information, the exercise information, and the diet information of another person, or character display on a map image in which the position information is used, can be performed.

Although a description was given for a configuration in which the biological exercise information detection display device 1 and the biological exercise information display device 200 include a display part 14 (214), it is also possible for the biological exercise information detection display device 1 and the biological exercise information display device 200 to not include a display part, and for character display to be executed using a separate display device.

In terms of privacy protection and similar issues, it is appropriate that a certain degree of restriction be placed on character display based on the biological information, the exercise information, and the diet information, or position display based on the position information, in regard to another person.

For example, a configuration can be envisaged in which, in an instance where the user of the biological exercise information display device 200 makes a request for character display or display of current position in relation to the user of the biological exercise information transmitting device 100, the server device 70 checks the preferences on the biological exercise information transmitting device 100, and only transmits biological information or character-related information to the biological exercise information display device 200 in an instance in which the user of the biological exercise information transmitting device 100 is shown to have consented.

A configuration is also possible in which in an instance in which the heart rate, blood pressure, or similar detected biological information is a normal value, the detected information is not used for character display, and it is only in an instance in which the value is physiologically abnormal that the corresponding biological information used in displaying the character, whereby character display is executed as a warning or a caution for the user or for another person.

According to an aspect of one embodiment, a biological exercise information display processing device, including a biological information obtaining part for obtaining biological information regarding a test subject; an exercise information obtaining part for obtaining exercise information regarding the test subject; a diet information obtaining part for obtaining diet information for the test subject; a display character obtaining part for obtaining a display character for the test subject; a display part for performing image display; and a control part for determining an expression or action of the display character based on the biological information, the exercise information, and the diet information; and performing a control so that an image display is performed on the display part according to the expression or action of the display character that has been determined.

According to the aspect described above, it is possible to present a biological exercise information display processing device in which the status of progress in improving lifestyle as described under the headings 1, 2, and 3 below is obtained from the biological information, the exercise information, and the diet information for the test subject; and displayed as a character display set up by the user themselves so as to be readily personalizable and easy to understand; whereby the test subject reviews their own progress status in a realistic manner for encouragement or reflection, or compares their own progress status with that of another person to boost competitiveness; and the test subject is able to increase their motivation and change their behavior.

The status of progress in improving lifestyle described above refers, for example, to the following.

1. Whether action targets that have been set to improve lifestyle are being achieved. Examples include whether the calorie intake in kilocalories is within the daily target calorie intake, or, with regard to exercise, whether the test subject is walking for 20 minutes a day.

2. The status of items regarding health management (e.g., weight, body fat percentage, BMI). An example is whether weight is decreasing.

3. Whether actions toward improving lifestyle (e.g., exercise, diet) are being sustained.

According to the aspect of the embodiment, the biological exercise information display processing device described above, further includes a storage part for storing information; wherein upon the biological information, the exercise information, and the diet information being obtained, the control part causes the storage part to store the obtained biological information, exercise information, and diet information, or character-related information representing expressions or actions of the display character determined based on the obtained biological information, exercise information, or diet information.

According to the aspect described above, the biological information, the exercise information, and the diet information for the test subject can be stored for a certain period of time.

According to the aspect of the embodiment, the biological exercise information display processing device described above, wherein the control part retrieves the biological information, the exercise information, and the diet information, or the character-related information, from the storage part under predetermined conditions, and performs display control of the display character using the biological information, the exercise information, and the diet information, or the character-related information, that have been retrieved.

According to the aspect described above, the biological information, the exercise information, and the diet information for the test subject are stored for a certain period of time. Therefore, by specifying a past point in time within the range in which the information is stored and causing the corresponding biological information, exercise information, and diet information to be extracted, it is possible to display the past status of the test subject using the display character.

According to the aspect of the embodiment, the biological exercise information display processing device described above, wherein the biological information is information regarding at least one of a pulse rate, heart rate, height, weight, BMI, abdominal girth, body fat percentage, blood pressure, glycemia, cholesterol, neutral fat, GOT, GPT, and γ-GTP.

According to the aspect described above, the biological information regarding the test subject can be readily obtained.

According to the aspect of the embodiment, the biological exercise information display processing device described above, wherein the exercise information is information regarding at least one of an exercise time, number of steps taken, calorie expenditure, and exercise type.

According to the aspect described above, the exercise information regarding the test subject can be readily obtained.

According to the aspect of the embodiment, the biological exercise information display processing device described above, wherein the diet information is at least one of a calorie intake, diet content, meal times, diet volume, nutritional balance, and dietary routines.

According to the aspect described above, the diet information for the test subject can be readily obtained.

According to another aspect of the embodiment, a biological exercise information processing system having a biological exercise information transmitting device and a biological exercise information display processing device, the biological exercise information processing system wherein the biological exercise information transmitting device includes a biological information detection part for detecting biological information regarding a test subject; an exercise information detection part for detecting exercise information regarding the test subject; and a transmitting part for transmitting/outputting the biological information and the exercise information; the biological exercise information display processing device includes a biological information obtaining part for obtaining the biological information regarding the test subject; an exercise information obtaining part for obtaining the exercise information regarding the test subject; a diet information obtaining part for obtaining diet information for the test subject; a display character obtaining part for obtaining a display character for the test subject; a display part for performing image display; a communication part for communicating information; and a control part for causing the communication part to execute a process for receiving the biological information and the exercise information; determining an expression or action of the display character based on the diet information and on the biological information and the exercise information received by the communication part; and performing a control so that an image display is performed on the display part according to the expression or action of the display character that has been determined.

According to the aspect described above, the user of the biological exercise information display device can know the current status of a test subject wearing the biological exercise information transmitting device using the display character image.

According to the aspect of the embodiment, the biological exercise information processing system described above, further having a server device including an accumulating part for accumulating the biological information, the exercise information, and the diet information; wherein the communication part of the biological exercise information display processing device transmits the biological information, the exercise information, and the diet information to the server device; the server device accumulates, in the accumulating part, the biological information, the exercise information, and the diet information that has been transmitted; and the communication part of the biological exercise information display processing device receives character-related information representing an expression or action of the display character, determined by the server device based on the biological information, the exercise information, and the diet information accumulated in the accumulating part.

According to the aspect described above, if the biological information and other information regarding the test subject who is wearing the biological exercise information transmitting device are stored for a certain period of time in the server device, by specifying a past point in time within the range in which the information is stored and causing the corresponding biological information and other information to be extracted, it is possible to display the past status of the test subject using a display character image.

What is claimed is:

1. A biological exercise information display processing device, comprising:
a biological information obtaining part that obtains biological information regarding a test subject;
an exercise information obtaining part that obtains exercise information regarding the test subject;
a diet information obtaining part that obtains diet information for the test subject;
a display character obtaining part that obtains a character representing the test subject;
a display part that displays the character; and
a control part that determines a point based on the exercise information and the diet information, and when a given number of points has been accumulated, performs an exchange process to exchange the points for an item of the character.

2. The biological exercise information display processing device according to claim 1, further comprising
a storage part that stores the biological information, the exercise information, the diet information, the point, and the item.

3. The biological exercise information display processing device according to claim 2, wherein
the control part retrieves the biological information, the exercise information, the diet information, the point, and the item, and displays the character with the item on the display part.

4. The biological exercise information display processing device according to claim 1, wherein
the biological information is information regarding at least one of a pulse rate, heart rate, height, weight, BMI, abdominal girth, body fat percentage, blood pressure, glycemia, cholesterol, neutral fat, GOT, GPT, and γ-GTP.

5. The biological exercise information display processing device according to claim 1, wherein
the exercise information is information regarding at least one of an exercise time, number of steps taken, calorie expenditure, and exercise type.

6. The biological exercise information display processing device according to claim 1, wherein
the diet information is at least one of calorie intake, diet content, meal times, diet volume, nutritional balance, and dietary routines.

7. The biological exercise information display processing device according to claim 1, wherein
the item includes clothes for the character.

8. A biological exercise information processing system comprising:
an information transmitting device; and
an information processing device,
the information transmitting device including
a biological information detection part that detects biological information regarding a test subject,
an exercise information detection part that detects exercise information regarding the test subject, and
a transmitting part that transmits the biological information and the exercise information,
the information processing device including
a communication part that obtains the biological information and the exercise information,
a diet information obtaining part that obtains diet information for the test subject,
a display character obtaining part that obtains a character representing the test subject,
a display part that displays the character, and
a control part that determines a point based on the exercise information and the diet information, and when a given number of points has been accumulated, performs an exchange process to exchange the points for an item of the character.

9. The biological exercise information display processing system according to claim 8, wherein
the display part displays the character with the item.

10. The biological exercise information display processing system according to claim 8, wherein
the biological information is information regarding at least one of a pulse rate, heart rate, height, weight, BMI, abdominal girth, body fat percentage, blood pressure, glycemia, cholesterol, neutral fat, GOT, GPT, and γ-GTP.

11. The biological exercise information display processing system according to claim 8, wherein
the exercise information is information regarding at least one of an exercise time, number of steps taken, calorie expenditure, and exercise type.

12. The biological exercise information display processing system according to claim 8, wherein
the diet information is at least one of calorie intake, diet content, meal times, diet volume, nutritional balance, and dietary routines.

13. The biological exercise information display processing system according to claim 8, wherein
the item includes clothes for the character.
